(19) United States
(12) Patent Application Publication
(10) Pub. No.: US 2005/0095629 A1
(43) Pub. Date: May 5, 2005

(54) Representative SNP selection method

(75) Inventors: Toshio Furuta, Tokyo (JP); Naoyuki Kamatani, Tokyo (JP); Yoshihiro Nakamura, Tokyo (JP); Toshikazu Ito, Tokyo (JP); Eisuke Inoue, Tokyo (JP)

Correspondence Address: OSTROLENK FABER GERB & SOFFEN, 1180 AVENUE OF THE AMERICAS, NEW YORK, NY, 100368403, US

(73) Assignees: NEC Corporation, Tokyo (JP); Mitsubishi Research Institute, Inc., Tokyo (JP); Japan Biological Informatics Consortium, Tokyo (JP)

(21) Appl. No.: 10/939,153

(22) Filed: Sep. 10, 2004

(30) Foreign Application Priority Data
Sep. 10, 2003 (JP) 317767/2003

(51) Int. Cl. C12Q 1/68; G06F 19/00; G01N 33/48; G01N 33/50

(52) U.S. Cl. 435/6; 702/20

(57) ABSTRACT

Multi-dimensional scaling is used to solve characteristic polynomials of similarity matrixes, in order to determine a minimal-number SNP set (htSNPs (haplotype-tagging SNPS)) for identifying an arbitrary number of haplotypes in blocks with strong linkage disequilibrium. Thus, unnecessary SNP typing in blocks with strong linkage disequilibrium can be avoided.

This application claims priority to prior application JP 2003-317767, the disclosure of which is incorporated herein by reference.

### BACKGROUND OF THE INVENTION

The present invention relates to a representative SNP selection method which can be widely applied to research using genomic polymorphism markers. The term “research using genomic polymorphism markers” as used here means association study, tailor-made medicine, and so forth.

As widely known, the 3 billion chemical base pairs which make up the human genome have been determined and reported. In the sequence of bases in human genes (DNA base sequence), a variation between individuals in a population which occurs with a frequency of at least around 1% is called a genetic polymorphism.

As also widely known, DNA (which is short for deoxyribonucleic acid) is almost entirely made up of the four bases of adenine (A), guanine (G), cytosine (C), and thymine (T).

Particularly, a polymorphism which exist regarding a single base is called a Single Nucleotide Polymorphism (SNP). SNPs are being given particular attention since they are polymorphisms which occur with a particularly high frequency. SNPs are estimated to exist at a rate of one every several hundred base pairs to 1,000 base pairs, and accordingly, 3 million to 10 million SNPs are assumed to exist in the human genome. Further, it is assumed that the great part of individual differences are determined by the differences in SNPs.

SNPs are thought to be easily employed as polymorphism markers, and development of equipment for clinical use is thought to be relatively easy, due to the following reasons.
(1) Several million copies or more exist within the genome.
(2) Determination is extremely easy, and results can be binarized, facilitating information processing.
(3) Technology for high-speed and massive-quantity SNP typing is in the process of being realized, and automated typing is not far away.

Revealing the effects of SNPs on genetic functions and genetic manifestation should lead to discovery of predispositions susceptible to certain diseases, better medical treatment tailored to such predispositions, and enabling selection and development of drugs and pharmaceuticals accordingly.

Of all SNPs, a set of multiple SNPs existing in proximity on a gene, which have been extracted as a genetic function block, is referred to as a haplotype.

Now, with X and Y each representing one of the bases adenine (A), guanine (G), cytosine (C), and thymine (T), there are three patterns for an SNP at one base site, e.g., X/X, X/Y, and Y/K.

There are primarily two approaches for genetic analysis, the functional approach and the genetic statistical approach. The former is an approach to analyze the functions of SNPs by molecular biology of the SNPs contained in genes. The latter is an approach to combine SNPs with clinical information and to extract the relation by genetic statistics. In the field of genetic statistics, methods for statistically uncovering potential relations between gene database and clinical databases are being studied. Genetic statistics allows the tendency of relations potentially existing in these database to be statistically uncovered.

The basis of genetic statistics is Mendel's Laws and linkage. It is crucial to understand Mendel's laws as laws pertaining to probability events. Mendel thought that there is behind the “phenotype” attributes of an individual organism which can be humanly recognized, a “genotype” which is not humanly recognizable, and that the “genotype” is stable only for the individual. He then discovered alleles (i.e., genes) which are stable across generations. The laws of Mendel can be understood to be laws relating to the above “phenotype”, “genotype”, and “alleles”. A locus is defined as a position where the three concepts can be defined. The laws of Mendel are summarized into the later-described “the law of segregation”, “the law of dominance”, and “the law of independent assortment”.

The nature observed at the level of individuals is expressed in terms of phenotype and trait. Traits are categories, and phenotypes are individual types observed in the traits. The concepts of locus, allele, and genotype must be defined along the line of Mendel in order to analyze the relation of individual traits.

A great number of loci are thought to exist for one mating population, generally stable in a species. The genotype is a stable unit for an individual. Each individual has one genotype at each locus. From a chromosomal expression perspective, a locus is a portion between minimal units where recombination occurs. From a molecular biology perspective, a locus is a single base. A locus is also referred to as a genetic locus.

While a genotype is stable on the individual level, a genotype has no stability across generations. Alleles are handed down in a stable manner from individual to individual, not genotypes. An allele is also called an allelomorph.

Mendel's law of segregation is a law regarding components making up genotypes which are stable while traversing generations. This law of segregation asserts that “a genotype is a combination of two alleles, and only one of the two alleles making up the genotype will be passed on to the next generation, at a probability which is equal to that of the other allele being passed on”. In other words, this law deals with probability. Accordingly, an allele is the smallest unit which can be handed from one generation to another in a stable manner. Of course, mutations destroy alleles, but Mendel's laws do not take mutations into consideration. At the chromosomal level, an allele is a locus on one strand of the chromosome, and in molecular biology is a type on one strand of the chromosome such as an SNP, STRP (short tandem repeat polymorphism), VNTR (variable number of tandem repeat), and so forth. For example, in an SNP, this would be one side of the base pair, such as T (thymine) or C (cytosine).

Mendel's law of domination asserts that “there are laws relating to the correlation between genotype and phenotype, and the phenotype of an individual is a function of the genotype”, indicating that there is dominance and non-dominance (i.e., recessive) in the function. At the chromosomal level, a genotype is a combination of two homologous loci existing at a homologous site on two homologous chromosomes. At the molecular biology level, a genotype is a combination of a polymorphism such as an SNP, or an STRP, a VNTR, or the like.

Linkage is an exception to Mendel's law of independent assortment. Mendel's law of independent assortment is a law relating to the relation between multiple loci, and asserts that “alleles at different loci are distributed to the child independently from one another”. At the chromosomal level, this is correct only regarding loci on different chromosomes. Two alleles on the same chromosome are physically joined, and accordingly enter the gamete together and are passed on to the child, meaning that the law of independent assortment is inapplicable. However, alleles on the same chromosome may not remain completely joined on to the next generation in some cases, since rearrangement of the joined alleles can occur due to crossing of the chromosome at the time of meiosis (recombination). The greater the distance between the loci is, the greatest the probability of recombination.

Morgan showed that Mendel's law of independent assortment does not necessarily apply to genes with different loci, thereby discovering linkage. However, the law of independent assortment is still correct regarding loci on different chromosomes, and moreover, it is crucial to understand that the law of independent assortment is axiomatic to the concept of linkage. Accordingly, the idea that Mendel's law of independent assortment was a mistake, is incorrect.

Now, the term independent means that the probability of two phenomena occurring simultaneously is expressed by the product of the probabilities of each phenomenon occurring individually. In genetic statistics, the term independent assortment does not mean being on the same chromosome, but rather is concerned with probability and statistics.

Defining linkage allows definition of combinations of the alleles at Locus 1 and Locus 2 (haplotype). A haplotype is a combination of alleles at linked loci in one gamete. A haplotype is preserved unless recombination occurs in meiosis. Recombination forms a new haplotype, which is passed on to the next generation by the gamete, and remains unchanged until recombination occurs again. The existence of a haplotype is a function of the haplotype in the gamete passed on from the parent and whether or not recombination has occurred between loci. The phenomenon of recombination occurs according to probability following the rate of recombination, so haplotype heredity can be thought of as a probability phenomenon having the recombination rate as a probability distribution function thereof. Linkage analysis calculates the order and distance of loci optimal for observation data relating to traits and genotypes of a lineage.

Each individual has two haplotypes, and the combination of the haplotypes is also referred to as a diplotype configuration.

The concept of linkage disequilibrium is paramount in genetic statistics. The difference between linkage and linkage disequilibrium must be correctly understood. The concept of linkage has been described above as an exception to Mendel's law of independent assortment, and that independent assortment is purely a concept of probabilistic concern. True linkage disequilibrium is a concept based on the assumption of linkage.

Genotypes exist at multiple linked loci, and if sufficient lineage information can be obtained, the diplotype configuration of the individual (the combination of two haplotypes) can be determined, as described above. A haplotype is a combination of individual alleles at loci on the same chromosome (i.e., linked). In case where lineage information does not exist, all information regarding the genetic information of the individual is the diplotype configuration of the individual.

Linkage disequilibrium is a law regarding distribution of haplotypes in a population. A diplotype configuration is a combination of two haplotypes. Therefore, the number of haplotypes in a population is double the number of individuals in that population.

Linkage disequilibrium is a phenomenon observed at two or more linked loci. In simple terms, linkage disequilibrium is a phenomenon wherein distribution of alleles between different loci is not independent, i.e., wherein the haplotype frequency deviates from the frequency obtained by multiplying the frequencies of alleles at the loci. Conversely, a state wherein the haplotype frequency equals the product of allele frequency (i.e., independent), is referred to as linkage equilibrium, and therefore, a state wherein linkage equilibrium is not realized is a state of linkage disequilibrium.

If a population is sufficiently great, and sufficient time has passed since all mutations have occurred, and further there is no change in the allele frequency, linkage equilibrium can be expected. However, in reality, no population is large enough, sufficient time has not passed, and moreover, the allele frequency changes. Further, in case where there is mixing between two populations which have each attained linkage equilibrium, strong linkage disequilibrium can be expected. Generally, almost all populations in the world exhibit strong linkage disequilibrium at close genetic distances. The farther the polymorphism, the smaller the degree of linkage disequilibrium is, Haplotypes involving new mutations are expected to exhibit the strongest linkage disequilibrium, and the greater the effective size of the population is, the smaller the linkage disequilibrium is expected to be.

The term “genetic (map) distance” is defined as “the value expected for the number of times of crossing between two loci”. Specifically, genetic distance is a concept defined based on cross-over, rather than recombination. The unit of measurement is Morgan (M). It should be understood however, that one cross-over does not necessarily occur per 1 M, rather, this is a concept of probability. Also, cross-over is a phenomenon in which partial crossing over occurs among homologous chromosomes during meiosis. Crossover is a chromosomal event which can be observed through the microscope, and refers to the action of crossing over. The number of times of cross-over per meiosis may not necessarily be one.

Linkage disequilibrium provides genetic statistics with many techniques. If the prediction of common-disease/common-variant/common-origin is correct, there should be a strong linkage disequilibrium between variations in disorders and variations in the surroundings, and linkage disequilibrium should be able to be used to find disorder-related sites.

Regions with strong linkage disequilibrium exist between regions where recombination has historically occurred. These regions are referred to as blocks, and are said to be around 10 to 15 kb (kilobase pair) in physical distance, though it is known that there are occasionally blocks around 100 kb or so. It is noted here that physical distance refers to the physical distance between two loci upon reading the base sequence.

In association studies, normally, polymorphism markers are disposed at equal intervals. “Association studies” as used here means case-control study or patient-oriented study, which is the easiest sort of study. As with linkage disequilibrium studies, association studies also handle populations of individuals with no lineage data, Samples of a patient population and a general population are collected separately, and difference in allele frequency therebetween, or difference in the frequency of individuals having particular alleles, is studied. This means that a greater number of markers are placed in a 100 kb block as compared to blocks of around 10 to 15 kb. For example, 30 or more SNPs may be typed for a massive block of 100 kb or more.

However, the cumulative frequency reaches or exceeds 95% with 5 or less haplotypes in the block region. Therefore, the number of SNPs needed for one block is at least three, from 2²=4 and 2³=8. There is absolutely no need for 30 SNPs in order to identify a mere five haplotypes at the most. Accordingly, this is a waste.

The following are documents on prior technology regarding the present invention. Japanese Unexamined Patent Publication (JP-A) No. 2003-14612 discloses a known method for analyzing SNPs by computer with fluorescent labeling, and discloses a analyzing system using a high-throughout SNP scoring system and a highly reliable SNP scoring system which is important for medical diagnosis. Japanese Unexamined Patent Publication (JP-A) No. 2002-300894 discloses a known method for typing SNPs, disclosing a typing technique for typing hundreds of thousands of SNP sites using a relatively small amount of genome DNA. Japanese Unexamined Patent Publication (JP-A) No. 2002-63175 discloses a known method for analyzing SNPs by computer to detect disorder-related SNPs, and discloses a technique for efficiently searching for SNPs with a high level of association with disorders from a great amount of data regarding SNPs along with the entire genome sequence. Japanese Unexamined Patent Publication (JP-A) No. 2002-272463 discloses a known method for determining the types of SNPs, and discloses a technique for easily determining the types of SNPs in a short time. Further, Japanese Unexamined Patent Publication (JP-A) No. 2003-52383 discloses a known method for deciding patterns, and discloses a method for identifying variations in the human genome and a technique for correlating these variations with genetic grounds for disorders and drug response.

The definition for “block” is disclosed by Mark J. Daly, John D. Rioux, Stephen F. Schaffner, Thomas J. Hudson, and Eric S. Lander, in “High-resolution haplotype structure in the human genome”, Nature genetics 2001 29; 229-232. Moreover, an EM algorithm serving as a method for estimating haploid frequency is known, as described by Laurent Excoffier and Montgomery Sltatkin in “Maximum-Likelihood Estimation of Molecular Haplotype Frequencies in a Diploid Population”, Mol. Biol. Evol. 1995 12(5); 921-927.

Each of the above mentioned Publications merely disclose a method of analyzing SNPs, but they do not disclose a method of avoiding unnecessary SNP typing.

### SUMMARY OF THE INVENTION

It is therefore an object of the present invention to realize a representative SNP selection method which is capable of avoiding unnecessary SNP typing in blocks with strong linkage disequilibrium.

Through careful and thorough study, the present Inventors have found that representative SNPs can be determined using multi-dimensional scaling for solving characteristic polynomials of similarity matrixes.

Namely, the present invention provides a method for selecting a minimal-number SNP set for identifying an arbitrary number of haplotypes in a block with strong linkage disequilibrium.

Specifically, according to a first aspect of the present invention, a method, for selecting representative SNPs by a computer system whereby a predetermined number of haplotypes can be identified from a plurality of haplotypes formed of a plurality of sets of SNPs existing close to one another on a gene in a block with strong linkage disequilibrium, comprises: a step for creating a similarity matrix from a haplotype matrix wherein each of the plurality of haplotypes are row vectors; a step for obtaining association between SNPs by solving characteristic equations of the similarity matrix; and a step for searching for the representative SNPs from association between the obtained SNPs.

In this method for selecting representative SNPs, alleles of the SNPs in the haplotype matrix may be represented by (0, 1), or by (1, −1). Also, a matrix obtained by centering the haplotype matrix is preferably used upon creating the similarity matrix. Further, the step for obtaining association between SNPs may further comprise a step for calculating the cumulative contribution ratio of eigenvalues obtained by solving the characteristic equations, and a step for calculating the distance between the SNPs from eigenvectors obtained by solving the characteristic equations; and the step for searching for representative SNPs may comprise a step for selecting the representative SNPs in the order that the distance is closer.

According to a second aspect of the present invention, a representative SNP selection system, whereby a predetermined number of haplotypes can be identified from a plurality of haplotypes formed of a plurality of sets of SNPs existing close to one another on a gene in a block with strong linkage disequilibrium, comprises: matrix creating means for creating a similarity matrix from a haplotype matrix wherein each of the plurality of haplotypes are row vectors; association calculating means for obtaining association between SNPs by solving characteristic equations of the similarity matrix; and searching means for searching for the representative SNPs from association between the obtained SNPs.

In this representative SNP selection system, alleles of the SNPs in the haplotype matrix may be represented by (0, 1), or by (1, −1). Also, a matrix obtained by centering the haplotype matrix is preferably used upon creating the similarity matrix. Further, the association calculating means may further comprise means for calculating the cumulative contribution ratio of eigenvalues obtained by solving the characteristic equations, and means for calculating the distance between the SNPs from eigenvectors obtained by solving the characteristic equations; and the searching means may comprise means for selecting the representative SNPs in the order that the distance is closer.

According to a third aspect of the present invention, a representative SNP selection program, whereby a computer can identify a predetermined number of haplotypes from a plurality of haplotypes formed of a plurality of sets of SNPs existing close to one another on a gene in a block with strong linkage disequilibrium, causes the computer to execute the following functions: a matrix creating function for creating a similarity matrix from a haplotype matrix wherein each of the plurality of haplotypes are row vectors; an association calculating function for obtaining association between SNPs by solving characteristic equations of the similarity matrix; and a searching function for searching for the representative SNPs from association between the obtained SNPs.

In this program, alleles of the SNPs in the haplotype matrix may be represented by (0, 1), or by (1, −1). Also, a matrix obtained by centering the haplotype matrix is preferably used upon creating the similarity matrix. Further, the association calculating function may further comprise a function for calculating the cumulative contribution ratio of eigenvalues obtained by solving the characteristic equations, and a function for calculating the distance between the SNPs from eigenvectors obtained by solving the characteristic equations; and the searching function may comprise a function for selecting the representative SNPs in the order that the distance is closer.

According to the present invention, the minimal number of SNPs capable of identifying haplotypes in a block with strong linkage disequilibrium can be determined.

### BRIEF DESCRIPTION OF THE DRAWINGS

FIG. 1 is a block diagram illustrating a hardware configuration of a computer system for realizing a representative SNP selection method according to the present invention; and

FIG. 2 is a flowchart for describing a program for executing a representative SNP selection method according to the present invention with a computer system shown in FIG. 1.

### DESCRIPTION OF PREFERRED EMBODIMENT

Detailed description will be made below regarding a selecting representative SNPs.

The method for selecting representative SNPs according to the present invention employs multi-dimensional scaling method by solving a characteristic equation for a similarity matrix, which is a kind of multivariate data analysis. First, description will be made regarding the multivariate data analyzing method.

Now, with an N-dimensional square matrix A, the scalar λ which satisfies the expression |A−λ·In|=0 is referred to as “eigenvalue of the matrix A”, and the expression |A−λ·In|=0 is referred to as “characteristic equation for the matrix A”. On the other hand, the left side of the expression, i.e., |A−λ·In|” is referred to as “characteristic polynomial”. The characteristic equation serves as an n-dimensional equation for the eigenvalue λ, and accordingly, the expression generally has n solutions. In case where a certain vector x other than zero vector can be determined so as to satisfy Ax=λᵢ·x (i=1 to n), the vector x is referred to as the eigenvector for the matrix A corresponding to the eigenvalue λₗ. In other words, the vector x which satisfies the characteristic equation (A−λᵢₗ)·x=0 is referred to as “eigenvector for the matrix A”. The single eigenvector xᵢ is obtained for each λₗ (i=1 to n).

The characteristic equation is employed in the representative SNP selecting method according to the present invention. The principal-component analysis is well-known as the multivariate data analyzing method using a characteristic equation. The principal-component analysis is a kind of multivariate data analysis for reducing the dimension of the high-dimensional data while suppressing loss of information as much as possible. With the principal-component analysis, the high-dimensional original data is converted to a linear combination of the variables independent one of another, each of which are referred to as “principal component”. With the principal-component analysis, the partial information is extracted from the information with regard to the population or the original data in order of importance thereof, such as the first principal component, the second component, and so on in the same way. In some cases, the original information is generally represented by a small number of the components. In this case, the following analysis is made using these small number of components. Thus, the principal-component analysis has the great advantage of simplifying analysis of the high-dimensional data while suppressing loss of the information thereof.

With the principal-component analysis, the relation between the variables of the data is obtained using a variance covariance matrix or a correlation matrix. Specifically, the principal-component analysis includes a step for transforming the variance covariance matrix or the correlation matrix to a diagonal matrix.

Next, description will be made regarding the variance covariance matrix. With the probability-variable vector as X=(X₁, X₂, . . . , Xₚ)′ (the symbol “′” represents transposition), μ represented by the following Expression 1 is referred to as “mean vector of the probability-variable vector X”.
μ=E(X)={E(X₁), E(X₂), . . . , E(Xₚ)}′ (1)

On the other and, the p×p symmetric matrix E represented by the following Expression 2 is referred to as “variance covariance matrix” or “variance matrix”.
E{(X−μ)(X−μ)′}=E{(Xᵢ−μᵢ)(Xⱼ−μⱼ)} (2)

It is noted here that the variances of the variables X₁ and X₂ will be represented by σ₁₁, and σ₂₂, respectively, and the covariance thereof will be represented by σ₁₂. Furthermore, ρ₁₂ represented by the following Expression 3 will be referred to as “correlation coefficient between X₁ and X₂”, which will be also represented by Corr(X₁, X₂). The correlation coefficient serves as an important measure for representing the linear association between the variables.

$${ρ}_{12} = \frac{{σ}_{12}}{\sqrt{{σ}_{11}} \sqrt{{σ}_{22}}} \qquad ( 3 )$$

For example, the variance covariance matrix Σ of the two-variable probability vector X=(X₁, X₂)′ is represented by the following Expression 4.

$$\sum = [ \begin{matrix}{σ}_{11} & {σ}_{12} \\ {σ}_{21} & {σ}_{22}\end{matrix} ] = [ \begin{matrix}{σ}_{1}^{2} & {σ}_{1} {σ}_{2} ρ \\ {σ}_{2} {σ}_{1} ρ & {σ}_{2}^{2}\end{matrix} ] \qquad ( 4 )$$

Note that σᵢ represents \sqrt{σᵢᵢ}, i=1, 2, and ρ=Corr(X1, X2).

Next, description will be made regarding the correlation matrix. With the variance of Xᵢ as var (Xᵢ), and with the covariance of Xᵢ and Xⱼ as cov(Xᵢ, Xⱼ), the correlation coefficient ρᵢⱼ and ρᵢᵢ, between Xᵢ and Xⱼ each of which are components of the p-dimensional variable probability vector X=(X₁, . . . , Xₚ)′ is represented by the following Expression 5.

$$\begin{matrix}{ρ}_{ij} = \frac{cov ( {X}_{i} , {X}_{j} )}{\sqrt{var ( {X}_{i} ) var ( {X}_{j} )}} = \frac{{σ}_{ij}}{\sqrt{{σ}_{ii} {σ}_{jj}}} & ( 5 ) \\ {ρ}_{ii} = 1 , {ρ}_{ij} = {ρ}_{ji} , \vert {ρ}_{ij} \vert < 1 & \end{matrix}$$

As described above, there is a close relation between the matrix P=(ρᵢⱼ) and the variance covariance matrix Σ=(σᵢⱼ). Here, i and j represent an integer of 1 to p. Here, the diagonal matrix having the diagonal components a₁, . . . , aₚ will be referred to as “diag(a₁, . . . , aₚ). Furthermore, D\sqrt{σ} and D⁻¹\sqrt{σ} will be represented by the following Expressions 6 and 7, respectively.

D_{\sqrt{σ}}=diag(\sqrt{σ₁₁}, . . . , \sqrt{σₚₚ}) (6)

$${D}^{- 1} \sqrt{σ} = {D}_{\frac{1}{\sqrt{σ}}} = diag ( \frac{1}{\sqrt{{σ}_{11}}} , … , \frac{1}{\sqrt{{σ}_{pp}}} ) \qquad ( 7 )$$

Using the Expressions 6 and 7, the following expressions 8 and 9 are obtained.
P=D⁻¹\sqrt{σ}ΣD⁻¹\sqrt{σ} (8)
Σ=D_{\sqrt{σ}}PD_{\sqrt{σ}} (9)
P is referred to as “correlation matrix of the p-dimensional variable probability vector” X.

Description will be made with regard to a specific example of two-dimensional-data calculation for clear understanding of calculation of the principal components. Let us say that the two-variable probability vector (X₁, X₂)′ is analyzed using the variance covariance matrix Σ represented by the following Expression 10.

$$\sum = [ \begin{matrix}{σ}_{11} & {σ}_{12} \\ {σ}_{21} & {σ}_{22}\end{matrix} ] \qquad ( 10 )$$

In this case, in general, the average vector may be transformed into μ=(μ₁, μ₂)′=0.

With the principal-component analysis, a linear combination Y=α₁·X₁+α₂·X₂ is determined such that the variation thereof represents a large part of the variation of the probability variable vector (X₁, X₂) with a given distribution as much as possible. Note that the average vector μ is transformed into 0, and accordingly, the linear combination Y passes through the origin. In other words, the coefficients α₁ and α₂ are determined such that the variance of Y exhibits the maximum value. Then, the maximum variance is compared to the total variation of the probability variable vector (X₁, X₂), and determination is made whether or not the obtained maximum variance is sufficient. If the maximum variance thus obtained is insufficient, the second linear combination (which will be referred to as “second principal component” hereafter) orthogonal to the first linear combination thus obtained (which will be referred to as “first principal component” hereafter) is calculated such that the variance of Y exhibits the maximum value.

With the normalized eigenvector corresponding to the maximum eigenvalue λ₁ obtained based upon the variance covariance matrix Σ as α₁, the first principal component of the probability vector X=(X₁, X₂)′ obtained based upon the variance covariance matrix Σ is determined to be the linear combination Y₁ represented by the following Expression 11.
Y₁=α₁₁X₁α₁₂X₂=α₁′X (11)

On the other hand, the second principal component is represented by the following Expression 12 using the normalized eigenvector α₂=(α₂₁, α₂₂) corresponding to the smaller (the second largest) eigenvalue λ2 calculated based upon the covariance variance matrix Σ.
Y₂=α₂₁X₁+α₂₂X₂=α₂′X (12)

In this case, the data of two variables is analyzed, and therefore, only two principal components exist. It is noted here that the following Expression 13 is obtained based upon the relation between the solutions and the coefficients.
σ₁₁+σ₂₂=λ₁+λ₂ (13)

The above Expression 13 means that the sum of the variances of the original variables X₁ and X₂ matches the sum of the variances of the principal components Y₁ and Y₂. The conservative value will be referred to as “total variance” hereafter. In this case, the ratio λ₁/(λ₁+λ₂) represents the ratio of the part to which the first principal component contributes, and accordingly, the ratio will be referred to as “the contribution ratio of the first principal component” hereafter.

Next, description will be made regarding calculation of the principal components in a case of analysis of p-variables data.

First, the variance covariance matrix Σ of the p-variables probability vector X=X(X₁, X₂, . . . , Xₚ)′ is calculated. The principal components of the p-variables probability vector X are obtained as p linear combinations each of which are formed of components independent one of another as represented by the following Expression 14. With the eigenvalues of the variance covariance matrix as λ₁, λ₂, . . . , λₚ (λ₁ □ λ₂ □ . . . □λₚ □O), the principal components are obtained as represented by the following Expression 14.
first principal component: Y₁=α₁₁X₁+α₁₂X₂+ . . . +α₁ₚXₚ=α₁′X
second principal component: Y=α₂₁X₁+α₂₂X₂+ . . . +α₂ₚXₚ=α₂′X (14)
p-th component: Yₚ=αₚ₁X₁+αₚ₂X₂+ . . . +αₚₚXₚ=αₚ′X

Note that αᵢ (i denotes an integer of 1 to p) denotes the eigenvector corresponding to the eigenvalue λᵢ, which is represented by the following Expression 15.
αᵢ′αᵢ=1, αᵢ′Σαⱼ=0, i≠j (15)
Furthermore, the following Expression 16 holds.
var(Yᵢ)=αᵢ′Σαᵢ=λᵢ, i=1, . . . , ρ (16)

The relation described above is represented by the following Expression 18 in the form of a matrix expression, using the following Expression 17.

$$Y = [ \begin{matrix}{Y}_{1} \\ {Y}_{2} \\ ⋮ \\ {Y}_{p1}\end{matrix} ] , A = [ {α}_{1} , {α}_{2} , ⋯ , {α}_{p} ] , A = [ \begin{matrix}{λ}_{1} & & & & 0 \\ & {λ}_{2} & & & \\ & & ⋰ & & \\ & & & ⋰ & \\ 0 & & & & {λ}_{p}\end{matrix} ] \qquad ( 17 )$$

Y=A′X, A′ΣA=A (18)

Note that the total variance is represented by the following Expression 19.
trΣ=σ₁₁+σ₂₂+ . . . +σₚₚ=λ₁+λ₂+ . . . +λₚ (19)

Here, the contribution ratio of the i-numbered principal component Yᵢ is represented by the following Expression 20.
λᵢ/(λ₁+ . . . +λₚ) (20)

Accumulated contribution ratio of k principal components Y₁, Y₂, . . . , Yₖ, is represented by the following Expression 21.
(λ₁+λ₂+ . . . +λₖ)/(λ₁+ . . . +λₚ), (k=1, 2, . . . p) (21)

As described above, the principal-component analysis has the advantage of representing a large part of the total variation (total variance) of the original data of X using a small number of the principal components, thereby facilitating analysis of high-dimensional data. The principal components used here have actual meaning, and may be named by the researcher.

Next, description will be made regarding the principal components obtained based upon the correlation matrix.

Description has been made regarding a method for calculating the principal components using the variance covariance matrix Σ of the probability vector X. In this event, the principal components changes corresponding to the unit of Xᵢ (i denotes an integer of 1 to p). Furthermore, in some cases, measurement is made with different scaling for each Xᵢ. In this case, the meaning of a linear combination thereof becomes less apparent, or becomes meaningless. Accordingly, the probability vector X is normalized as represented by the following Expression 22.

$${Z}_{i} = \frac{{X}_{i} - ɛ ( {X}_{i} )}{\sqrt{var ( {X}_{i} )}} = \frac{{X}_{i}}{\sqrt{{σ}_{ii}}} , i = 1 , ⋯ , p \qquad ( 22 )$$

Then, the principal-component analysis is preformed by the use of the variance covariance matrix of Z=Z(Z₁, Z₂, . . . , Zₚ)′. In this case, the variance covariance matrix of Z matches the correlation matrix P=(ρᵢⱼ). The principal components are calculated based upon the matrix P and the eigenvectors of P in the same way as with the calculating method using Σ. Note that it is needless to say that the eigenvalues and the eigenvectors calculated based upon P do not match those calculated based upon Σ, accordingly, the principal components thus obtained do not match those obtained using Σ.

With the eigenvalues of P as δ₁, δ₂, . . . , δₚ (δ₁ □δ₂ □ . . . □δₚ □0), the total variance is represented by the following Expression 23.
trP=p=δ₁+δ₂+ . . . +δₚ (23)

Here, the contribution ratio of the i-numbered principal component is represented by the following Expression 24, and the accumulated contribution ratio of the k principal components U₁, U₂, . . . , Uₖ, is represented by the following Expression 25.
δᵢ/p, i=1 . . . , p (24)
(δ₁+ . . . +δₖ)/p, k=1 . . . , p (25)

The analyzing method using the correlation matrix P instead of the variance covariance matrix Σ has one disadvantage. Namely, normalization of the original variables used in the analysis applies the same weight to all the variables, may leading to distortion of the measurement results. That is to say, in analogy, measurements accurately stating the fact that “the head is a part of the body”, would not be precisely represented,.

Furthermore, the multi-dimensional scaling method using the characteristic equation of the similarity matrix is also well-known. With the multi-dimensional scaling method, the reasonable average relation between the elements of the data is calculated in the form of the multi-dimensional (at as low a dimension as possible) spatial layout based upon a great number of data sets of the subjective relative distance between the elements which can be discriminated one from another, with the researcher having no clear understanding of the relation between the elements. Note that in some cases, the researcher does not clearly discriminate between the elements.

With the method according to the present invention, the relation between the SNPs is obtained by solving the characteristic equation. Thus, the representative SNPs are detected.

Let us say that the data which has been subjected to typing of the SNPs at the L loci is analyzed for N/2 individuals. The allele at each locus is represented as follows. Specifically, the allele as to the gene A is denoted by a, for example. The data which is to be analyzed is represented in the form of an N×L matrix wherein the row represents the sample number of the chromosome, and the column represents the locus. The N×L matrix will be denoted by “M”, and will be referred to as “haplotype matrix” hereafter. The (i, j) component mij of the N×L matrix (haplotype matrix) represents the allele of the i-numbered haplotype and j-numbered locus. The i-numbered haplotype is represented by the row vector mi of the N×L matrix (haplotype matrix). It is noted here that the N×L matrix (haplotype matrix) M and the row vector mi thereof are represented by the following Expressions 26 and 27, respectively.
M=(m₁, m₂, . . . , m_{N})′, (26)
mᵢ=(mᵢ₁, mᵢ₂, . . . , m_{iL})′, (i=1, . . . , N) (27)

Let us say that a population which is to be analyzed has S kinds of haplotypes. Each haplotype is represented by the i-numbered haplotype hᵢ (i denotes an integer of 1 to S). Let us say that the first haplotype h1 has the highest frequency, and the S-numbered haplotype has the smallest frequency. With the row vector mᵢ (i denotes an integer of 1 to N) which matches the first haplotype h₁, all the alleles at the corresponding loci are set to A for convenience of the following calculation. At the time of the following matrix calculation, A and a are set to suitable values such as A=0 and a=1.

The present invention proposes a selecting method for selecting the representative SNPs in a haplotype block. With the selecting method proposed here, analysis is made using the characteristic equation as described above. The principal-component analysis described above is well known, which is a kind of the multi-variable data analyzing method using the characteristic equation. With the principal-component analysis, the relation between the variables is obtained using the variance covariance matrix or the correlation matrix described above. As another method, the multi-dimensional scaling method using the characteristic equation of the similarity matrix is also well-known. In the method according to the present invention, the relation between the SNPs is obtained by solving the characteristic equation. Thus, the representative SNPs are detected.

First, the N×L matrix (haplotype matrix) M and the column vector mⱼ* are represented by the following Expression 28 and 29, respectively.
M=(m₁*, m₂*, m_{L}*), (28)
mⱼ*=(m₁ⱼ, M₂ⱼ, . . . , M_{Nj}) (j=1, . . . , L) (29)

The column vector mⱼ* represents the allele patterns at j-numbered locus for N individuals. The multiple same allele patterns are represented by the single same allele pattern without loss of information. In this event, the allele pattern vectors are projected onto a one-dimensional unit vector u, and the similarity between the SNPs is determined based upon the positional relation on the unit vector. Under this circumstance, the unit vector is determined such that the allele pattern vector projected thereon exhibits the maximum variance.

Let us say that analysis is made using the N×L matrix subjected to centering. The unit vector is determined such that the variance of the allele vector patterns projected thereon exhibits the maximum value. It is noted here that the aforementioned centering means that each row vector of the matrix M is transformed such that the average of the components thereof matches zero, thereby allowing the researcher to handle the variation as variance.

The allele pattern vectors projected onto the unit vector u are represented by (M·u), and the variance thereof, i.e., the sum of squares thereof is represented by (u′·M′·M·u). Note that the matrix (M′·M) is referred to as a “similarity matrix”. In this event, the unit vector u described above is determined by solving the characteristic equation represented by the following Expression 30 under the condition of u′·u=1.
M′Mu=1u (30)

The characteristic equation represented by the Expression 30 is solved, so that the eigenvalues λ and the corresponding eigenvectors u are obtained. Then, the contribution ratio is calculated for each eigenvector in the same way as with the principal-component analysis. With the eigenvalues thus obtained as λ₁, λ₂, . . . , λ_{L} (λ₁>λ₂> . . . >λ_{L}), the contribution xᵢ for each eigenvalue xᵢ (i denotes an integer of 1 to L) is represented by the following Expression 31.

$${x}_{i} = {λ}_{i} / {\sum }_{j = 1}^{L} {λ}_{j} \qquad ( 31 )$$

On the other hand, the accumulated contribution ratio of the first eigenvalue through the i-ordered eigenvalue, i.e., αᵢ, is represented by the following Expression 32.

$${α}_{i} = {\sum }_{k = 1}^{i} {λ}_{k} / {\sum }_{j = 1}^{L} {λ}_{j} \qquad ( 32 )$$

The rank of the L-dimensional square matrix (M′·M) which is also referred to as a “similarity matrix” matches the number of the kinds of the haplotypes in the region formed of L loci which are to be analyzed. The accumulated contribution ratio of the first eigenvalue through the i-ordered eigenvalue, i.e., αᵢ, serves as a scale for representing the number of the representative SNPs which are to be taken for the following analysis. Furthermore, the accumulated contribution ratio αᵢ is used for determining the dimensional number of the unit-eigenvector space for analyzing the positional relation of the SNPs. Specifically, with the present analyzing method, if the accumulated contribution ratio CL of the two or three eigenvalues exhibits 0.8 or more, the researcher analyzes the positional relation between the SNPs in the two-dimensional unit-eigenvector space.

Now, the first eigenvalue λ₁ and the second eigenvalue λ₂ will be represented by u₁ and u₂, respectively. With the present analyzing method, the distance is calculated between the desired SNPs at the corresponding loci in the two-dimensional unit-eigenvector space formed of the eigenvectors u₁ and u₂. The data of the distance between the SNPs is represented in the form of a matrix, which will be referred to as the distance matrix D hereafter. Note that the component dᵢⱼ of the distance matrix D represents the distance between the SNPs at the corresponding loci i and j.

As described above, the loci which exhibit similar variation in the allele between haplotypes are represented by similar positions in the eigenvector space. For example, the locus i and the locus j which exhibit completely the same variation between all the haplotypes are represented by the same position in the eigenvector space. Specifically, in this case, the distance dij between the positions corresponding to the locus i and the locus j is zero in the eigenvector space. On the other hand, let us say that the locus i and the locus j exhibit different variation only in a low-frequency haplotype. In this case, the distance dij between the positions corresponding to the locus i and the locus j exhibits a small value in the eigenvector space. Therefore, the small distance dij between the positions corresponding to the locus i and the locus j in the eigenvector space means that the alleles positioned at the aforementioned loci exhibit similar variation between the haplotypes.

With the present analyzing method, the representative SNPs are selected using the distance matrix D. In case where analysis must be made with the absolutely whole haplotype information regardless of the frequency thereof, the loci in which the distance dᵢⱼ therebetween exhibits zero are represented by the single representative locus, thereby reducing the number of the variables (loci) while maintaining the original information, which is the object of the present invention. Note that the loci in which the distance dᵢⱼ therebetween exhibits zero can be easily detected by making comparison between the independent vectors of the N×L matrix M in the locus direction. On the other hand, in case where analysis should be made with a predetermined number of the SNPs, the loci is represented by the single locus in order of smaller distance between the positions in the eigenvector space. The processing is consecutively performed until the number of the loci each of which have been selected as a representative locus is reduces to a predetermined number of the SNPs.

In this example, the distance matrixes D formed for two patterns of (A=0, a=1) and (A=1, a=−1) were estimated giving consideration to estimation results affected by settings of the alleles A and a. That is to say, variation in the settings of the alleles A and a varies the components of the L-dimensional square matrix (M′·M), leading to variation in the eigenvalues and eigenvectors of the L-dimensional square matrix (M′·M) (similarity matrix). Accordingly, it is important to estimate the distance matrix D with variation in the settings of the alleles A and a.

In case of the settings of (A=0, a=1), the number of the same kind of haplotypes having the allele “a” in the row vectors of the N×L matrix M is important for the L-dimensional square matrix (M′·M) (similarity matrix). On the other hand, the number of the same kind of the haplotypes having the allele “A” in the row vectors of the N×L matrix M does not affect the L-dimensional square matrix (M′·M) (similarity matrix),

However, in case of the settings of (A=1, a=−1), a single haplotype having the allele “A” adds “1” to the contribution of the same kinds of the haplotypes to the similarity matrix. On the other hand, a single haplotype having the allele “a” adds “−1” to the contribution of the same kinds of the haplotypes to the similarity matrix. Accordingly, with the settings of (A=1, a=−1), it is assumed that the positions of the SNPs in the eigenvector space suitable for discriminating the haplotypes with generally the same frequency would be calculated so as to exhibit generally the same distance therebetween. Thus, the settings of (A=1, a=−1) has the advantage of precise estimation of the SNPs without adverse effects due to the high-frequency haplotypes or alleles.

### EXAMPLE 1

Subsequently, description will be made regarding an Example (calculation example) of the present invention.

Description will be made regarding the advantage of the analyzing method according to the present invention using the data of “BLOCK 4” disclosed in the above Reference by Mark J. Daily et al. The BLOCK 4 includes eleven SNPs. All the minor allele frequencies are 10% or more. The data has been subjected to the TDT analysis. Accordingly, data sampling was taken only from founders, in order to eliminate members of the same family.

The aforementioned TDT is an abbreviation of “Transmission disequilibrium test” , which is a markedly effective method having an extremely simple mechanism. While TDT analysis requires a great number of sub-families, a single family needs to include at least one patient, unlike non-parametric analysis. The patient has inherited half of the genes from each parent. Therefore, one of the two alleles of a parent is passed on to a child with the same probability. Namely, one of the two alleles is passed on to a child with a probability of 0.5.

TDT analysis is an analyzing method for determining which allele has been passed on to the patient from the parent. If the disorder of the patient has no relation to the marker locus, either of the alleles at the marker locus of the patient is passed on to the patient with the same probability. With the TDT analysis, deviation of the aforementioned probability is statistically estimated. In case where the researcher can discriminate all the four alleles of the parents, calculation is easily performed. However, in general, there is the need to make statistical study. That is to say, while the TDT analysis has a simple mechanism based upon the fact that the gene passed on to the patient from the parent has relation to the disorder thereof, in actuality, there is the need to make statistical study for a great number of lineages. The reason why such statistical study must be made is that linkage disequilibrium occurs between the disorder locus and the marker locus in the population formed of the great number of lineages different one from another. The TDT analysis has the advantage of resolving the structural problem to a certain degree, which occurs in the case-control study. That is, the controlled allele population used here is an allele population having no relation to disorder inherited from the parents. The alleles are not sampled from another population independent of the population which is to be analyzed.

The TDT analysis has been proposed primarily for detecting linkage between the marker locus assumed to be a locus having relation to the disorder and the disorder locus based upon the family correlation analysis. In other words, with the TDT analysis, examination is made whether or not true linkage disequilibrium occurs between the marker locus and the disorder locus. It is noted here that the aforementioned “true linkage disequilibrium” means that there is linkage between the marker locus and the disorder locus, as well as correlation therebetween. On the other hand, “false linkage disequilibrium” means that there is correlation between the marker locus and the disorder locus without linkage therebetween,

The following Table 1 shows an input data example.

TABLE 1

| Sample | L1 | L2 | L3 | L4 | L5 |
|---|---|---|---|---|---|
| 430 | T/A | G/A | G/C | C/A | T/A |
| 431 | T/T | A/A | C/C | A/A | A/A |
| 438 | T/T | A/A | C/C | A/A | A/A |
| 444 | T/T | A/A | C/C | A/A | A/A |
| 543 | T/T | A/A | C/C | A/A | A/A |
| 513 | T/T | A/A | C/C | A/A | A/A |
| 573 | T/A | G/A | G/C | C/A | T/A |
| 574 | T/T | A/A | C/C | A/A | A/A |
| 1011 | T/T | A/A | C/C | A/A | A/A |
| 641 | T/T | A/A | C/C | A/A | A/A |
| 999 | T/A | G/A | G/C | C/A | T/A |

The first row in Table 1 shows a “sample 430” in which the genotype T/A is positioned at the first locus L1, the genotype G/A is positioned at the second locus L2, and so on in the same way. The other sampled data sets are arrayed in the same way, whereby the data table is formed with the same number of rows as the number of the samples.

In general, the observed data is obtained in the form of genotype data, and therefore, there is the need to estimate the corresponding haplotypes. In this case, while one of the alleles at the first locus L1, e.g. thymine (T), is determined to be 0, the other one, e.g., adenine (A) is determined to be 1, for example. In case where the observed data is obtained in the form of the haplotype data, there is no need to perform such estimation processing. The haplotype-frequency estimation is made using the EM algorithm described in the above Reference by Laurent Excoffier and Montgomery Sltatkin. The following Table 2 shows the estimation results.

TABLE 2

| | | | Expected |
|---|---|---|---|
| | Haplotype | Frequency | Number |
| h₁ | 00000000000 | 0.441 | 228 |
| h₂ | 011111111111 | 0.199 | 103 |
| h₃ | 01110000010 | 0.182 | 94 |
| h₄ | 11110001111 | 0.116 | 60 |
| h₅ | 01111111110 | 0.014 | 7 |
| h₆ | 00000000010 | 0.011 | 5 |
| | | 0.963 | 497 |

The locus number will be denoted by L₁, L₂. . . , L₁₁, in order from the left in Table 2. In Table 2, all the alleles of the first haplotype h₁ having the highest frequency are represented by “0” for convenience of the following description. Note that “0” and “1” represent different alleles for each locus.

With the present example, let us say that the represented SNPs are selected from the first locus through the eleventh locus for discriminating the haplotypes having frequency of 1 % or more. The estimation results of the haplotype frequency are shown in Table 2. As shown in Table 2, there are two kinds of haplotypes with haplotype frequency of around 1%. On the other hand, while there are several kinds of haplotypes with haplotype frequency of 1% or less, such haplotypes are ignored. Accordingly, the N×L matix (haplotype matrix) M is formed of the expected number of the haplotypes arrayed as row vectors. Note that the expected number is calculated by multiplying the haplotype frequency by twice the number of the samples.

In the present example, the N×L matrixes (haplotype matrix) M formed with variation of the settings of “A” and “a” corresponding to the alleles are estimated.

First, description will be made regarding a case of the settings of (A=0, a=1).

The following Table 3 shows the L-dimensional square matrix (similarity matrix) (M′·M).

TABLE 3

| | | | | | | | | | | |
|---|---|---|---|---|---|---|---|---|---|---|
| 60 | 60 | 60 | 60 | 0 | 0 | 0 | 60 | 60 | 60 | 60 |
| 60 | 264 | 264 | 264 | 110 | 110 | 110 | 170 | 170 | 264 | 163 |
| 60 | 264 | 264 | 264 | 110 | 110 | 110 | 170 | 170 | 264 | 163 |
| 60 | 264 | 264 | 264 | 110 | 110 | 110 | 170 | 170 | 264 | 163 |
| 0 | 110 | 110 | 110 | 110 | 110 | 110 | 110 | 110 | 110 | 103 |
| 0 | 110 | 110 | 110 | 110 | 110 | 110 | 110 | 110 | 110 | 103 |
| 0 | 110 | 110 | 110 | 110 | 110 | 110 | 110 | 110 | 110 | 103 |
| 60 | 170 | 170 | 170 | 110 | 110 | 110 | 170 | 170 | 170 | 163 |
| 60 | 170 | 170 | 170 | 110 | 110 | 110 | 170 | 170 | 170 | 163 |
| 60 | 264 | 264 | 264 | 110 | 110 | 110 | 170 | 170 | 269 | 163 |
| 60 | 163 | 163 | 163 | 103 | 103 | 103 | 163 | 163 | 163 | 163 |

Then, the haplotype matrix M is centered, thereby allowing the researcher to handle the variation as variance. The calculation results are shown in the following Table 4.

TABLE 4

| | | | | | | | | | | |
|---|---|---|---|---|---|---|---|---|---|---|
| 52.8 | 28.1 | 28.1 | 28.1 | −13.3 | −13.3 | −13.3 | 39.5 | 39.5 | 27.5 | 40.3 |
| 28.1 | 123.8 | 123.8 | 123.8 | 51.6 | 51.6 | 51.6 | 79.7 | 79.7 | 121.1 | 76.4 |
| 28.1 | 123.8 | 123.8 | 123.8 | 51.6 | 51.6 | 51.6 | 79.7 | 79.7 | 121.1 | 76.4 |
| 28.1 | 123.8 | 123.8 | 123.8 | 51.6 | 51.6 | 51.6 | 79.7 | 79.7 | 121.1 | 76.4 |
| −13.3 | 51.6 | 51.6 | 51.6 | 85.7 | 85.7 | 85.7 | 72.4 | 72.4 | 50.5 | 66.9 |
| −13.3 | 51.6 | 51.6 | 51.6 | 85.7 | 85.7 | 85.7 | 72.4 | 72.4 | 50.5 | 66.9 |
| −13.3 | 51.6 | 51.6 | 51.6 | 85.7 | 85.7 | 85.7 | 72.4 | 72.4 | 50.5 | 66.9 |
| 39.5 | 79.7 | 79.7 | 79.7 | 72.4 | 72.4 | 72.4 | 111.9 | 111.9 | 78.0 | 107.2 |
| 39.5 | 79.7 | 79.7 | 79.7 | 72.4 | 72.4 | 72.2 | 111.9 | 111.9 | 78.0 | 107.2 |
| 27.5 | 121.1 | 121.1 | 121.1 | 50.5 | 50.5 | 50.5 | 78.0 | 78.0 | 123.4 | 74.8 |
| 40.3 | 76.4 | 76.4 | 76.4 | 66.9 | 66.9 | 66.9 | 107.2 | 107.2 | 74.8 | 109.5 |

The following Table 5 shows the eigenvalues of the L-dimensional square matrix (similarity matrix) (M′·M).

TABLE 5
Eigenvalue (A = 0, a = 1)

| | | Contribution Ratio |
|---|---|---|
| | Eigenvalue | (Cumulative) |
| 1 | 40.310 | 0.581 (0.581) |
| 2 | 14.388 | 0.207 (0.789) |
| 3 | 10.543 | 0.152 (0.940) |
| 4 | 2.190 | 0.032 (0.972) |
| 5 | 1.933 | 0.028 (1.000) |

As can be understood from Table 5, the accumulated contribution ratio of the three eigenvalues exhibits approximately 94%, and the accumulated contribution ratio of the four eigenvalues exhibits approximately 97%. Accordingly, estimation is made that the number of the representative SNPs is approximately three or four for discriminating the haplotypes in the block data.

The following Table 6 shows the eigenvectors corresponding to the first and second eigenvalues.

TABLE 6
Eigenvalue (A = 0, a = 1)

| | First | Second |
|---|---|---|
| Locus | Eigenvector | Eigenvector |
| L₁ | 0.0934 | −0.1822 |
| L₂ | 0.3884 | −0.2768 |
| L₃ | 0.3884 | −0.2768 |
| L₄ | 0.3884 | −0.2768 |
| L₅ | 0.2052 | 0.4113 |
| L₆ | 0.2052 | 0.4113 |
| L₇ | 0.2052 | 0.4113 |
| L₈ | 0.2986 | 0.2291 |
| L₉ | 0.2986 | 0.2291 |
| L₁₀ | 0.3896 | −0.2837 |
| L₁₁ | 0.2867 | 0.2095 |

As can be understood from Table 6, the loci can be classified into the locus sets (L₂, L₃, L₄), (L₅, L₆, L₇), and (L₈, L₉) each of which include the loci represented by the same position in the eigenvector space formed of the first and second eigenvectors. Note that as can be understood from Table 2, the loci in the same locus set thus classified exhibit the same variation between the haplotypes.

As described above, in case where analysis must be made with the absolutely whole haplotype information regardless of the frequency thereof, the loci in which the distance d(i, j) therebetween exhibits zero are represented by the single representative locus, thereby reducing the number of the variables (loci) while maintaining the original information, which is the object of the present invention. With the present example, the six SNPs of the first locus L1, the second locus L₂, the fifth locus L₅, the eighth locus L₈, the tenth locus L₁₀, and the eleventh locus L₁₁, are determined to be the representative SNPs, for example.

In case where analysis should be made with a predetermined number of the SNPs, the loci is represented by the single locus in order of smaller distance between the positions in the eigenvector space. The processing is consecutively performed until the number of the loci each of which have been selected as a representative locus is reduces to a predetermined number of the SNPs. With the present example, let us say that the number of the representative SNPs is reduced to four.

The distance between the locus positions is calculated in the two-dimensional eigenvector space for each locus pair. The calculation results are shown in the following Table 7.

TABLE 7
Distance (A = 0, a = 1)

| | L₂ | L₅ | L₈ | L₁₀ | L₁₁ |
|---|---|---|---|---|---|
| L₁ | 0.310 | 0.604 | 0.460 | 0.313 | 0.437 |
| L₂ | | 0.712 | 0.514 | 0.007 | 0.497 |
| L₅ | | | 0.205 | 0.719 | 0.218 |
| L₈ | | | | 0.521 | 0.023 |
| L₁₀ | | | | | 0.504 |

As described above, the locus sets (L₂, L₃, L₄), (L₅, L₆, L₇), and (L₈, L₉) are represented by the same positions in the eigenvector space, respectively. Accordingly, these locus sets are represented by the single loci L₂, L₅, and L₈, respectively.

As can be understood from Table 7, the distance between the second locus L₂ and the tenth locus L₁₀ exhibits the smallest value of 0.007. As can be understood from Table 2, the second locus L₂ and the tenth locus L₁₀ exhibit different variation only between the first haplotype h₁ and the sixth haplotype h₈. Specifically, the SNPs at the second locus L₂ and the tenth locus L₁₀ may be selected for discriminating the sixth haplotype h₆ having the smallest frequency. Accordingly, it can be understood that in case where the two loci of the second locus L₂ and the tenth locus L₁₀ are represented by the single one thereof, the information is lost with regard to the sixth haplotype having small frequency of 1.1%, but the number of the representative SNPs is reduced. In case where the second locus L₂ is omitted, discrimination cannot be made between the sixth haplotype h₆ and the third haplotype h₃. On the other hand, in case where the tenth locus L₁₀ is omitted, discrimination cannot be made between the sixth haplotype h₆ and the first haplotype h₁.

Specifically, if the second locus L₂ is omitted, the representative SNPs are determined to be at the five loci of the first locus L₁, the fifth locus L₅, the eighth locus L₈, the tenth locus L₁₀, and the eleventh locus L₁₁. On the other hand, if the tenth locus L₁₀ is omitted, the representative SNPs are determined to be at the five loci of the first locus L₁, the second locus L₂, the fifth locus L₅, the eighth locus L₈, and the eleventh locus L₁₁. In the present example, the tenth locus L₁₀ is omitted.

As can be understood from Table 7, the distance between the eighth locus L₈ and the eleventh locus L₁₁, exhibits the second smallest value of 0.023. As can be understood from Table 2, the eighth locus L₈ and the eleventh locus L₁₁ exhibit different variation only between the second haplotype h₂ and the fifth haplotype h₅. Specifically, the SNPs at the eighth locus L₈ or the eleventh locus L₁₁ may be selected for discriminating the fifth haplotype h₅ having the second smallest frequency. Therefore, it can be understood that in case where the two loci of the eighth locus L₈ and the eleventh locus L₁₁, are represented by the single one thereof, the information is lost with regard to the fifth haplotype, but the number of the representative SNPs is reduced. If the eleventh locus L₁₁ is omitted, discrimination cannot be made between the fifth haplotype h₅ and the second haplotype h₂. On the other hand, even if the eighth locus L₈ is omitted, the fifth haplotype h₅ can be discriminated from any of the other haplotypes.

Accordingly, in the present example, the eleventh locus L₁₁ is omitted, so that the four loci of the first locus L₁, the second locus L₂, the fifth locus L₅, and the eighth locus L₈, are selected as the representative SNPs. This processing reduces the number of the representative SNPs to a predetermined number. That is to say, in the present example, the four SNPs are typed in order to discriminate the first through fourth haplotypes h1 through h₄.

As can be understood from Table 7, the distance between the fifth locus L₅ and the eighth locus L₈ exhibits the third smallest value. As can be understood from Table 2, the fifth locus 5 and the eighth locus L₆ exhibit different variation only between the fourth haplotype having the fourth smallest frequency and any of the other haplotypes. However, the fourth haplotype h₄ is a common haplotype having frequency of 10% or more, and therefore, the fourth haplotype must be discriminated. Further, the frequency of the fourth haplotype h₄ is approximately ten times as high as the frequency of the fifth haplotype h₅. On the other hand, the distance d(5, 8) corresponding to discrimination of the fourth haplotype h4 exhibits ten times as great as the distance d(8, 11) corresponding to discrimination of the fifth haplotype h5. As can be understood from the relation between the frequency and the distance described above, the distance between the haplotypes for discrimination of a desired haplotype corresponds to the frequency of the haplotype. Accordingly, the distance can be used as a scale for the number of the representative SNPS.

As described above, in the present example, the SNP sets each of which are formed of SNPs having the same distance are represented by the single SNP thereof in order of smaller distance, whereby the information is lost with regard to haplotypes in order of smaller frequency.

Next, description will be made regarding a case of the settings of (A=1, a=−1).

Now, description will be omitted regarding the same calculation as with a case of the settings of (A=0, a=1), and description will be made regarding particular calculation.

The following Table 8 shows the L-dimensional square matrix (similarity matrix) (M′·M).

TABLE 8

| | | | | | | | | | | |
|---|---|---|---|---|---|---|---|---|---|---|
| 497 | 89 | 89 | 89 | 157 | 157 | 157 | 277 | 277 | 79 | 291 |
| 89 | 497 | 497 | 497 | 189 | 189 | 189 | 309 | 309 | 487 | 295 |
| 89 | 497 | 497 | 497 | 189 | 189 | 189 | 309 | 309 | 487 | 295 |
| 89 | 497 | 497 | 497 | 189 | 189 | 189 | 309 | 309 | 487 | 295 |
| 157 | 189 | 189 | 189 | 497 | 497 | 497 | 377 | 377 | 179 | 363 |
| 157 | 189 | 189 | 189 | 497 | 497 | 497 | 377 | 377 | 179 | 363 |
| 157 | 189 | 189 | 189 | 497 | 497 | 497 | 377 | 377 | 179 | 363 |
| 277 | 309 | 309 | 309 | 377 | 377 | 377 | 497 | 497 | 299 | 483 |
| 277 | 309 | 309 | 309 | 377 | 377 | 377 | 497 | 497 | 299 | 483 |
| 79 | 487 | 487 | 487 | 179 | 179 | 179 | 299 | 299 | 497 | 285 |
| 291 | 295 | 295 | 295 | 363 | 363 | 363 | 483 | 483 | 285 | 497 |

The calculation results following centering are shown in Table 9.

TABLE 9

| | | | | | | | | | | |
|---|---|---|---|---|---|---|---|---|---|---|
| 211.0 | 112.5 | 112.5 | 112.5 | −53.1 | −53.1 | −53.1 | 157.9 | 157.9 | 110.1 | 161.3 |
| 112.5 | 495.1 | 495.1 | 495.1 | 206.3 | 206.3 | 206.3 | 318.8 | 318.8 | 484.4 | 305.7 |
| 1125 | 495.1 | 495.1 | 495.1 | 206.3 | 206.3 | 206.3 | 318.8 | 318.8 | 484.4 | 305.7 |
| 112.5 | 495.1 | 495.1 | 495.1 | 206.3 | 206.3 | 206.3 | 318.8 | 318.8 | 484.4 | 305.7 |
| −53.1 | 206.3 | 206.3 | 206.3 | 342.6 | 342.6 | 342.6 | 289.5 | 289.5 | 201.9 | 267.7 |
| −53.1 | 206.3 | 206.3 | 206.3 | 342.6 | 342.6 | 342.6 | 289.5 | 289.5 | 201.9 | 267.7 |
| −53.1 | 206.3 | 206.3 | 206.3 | 342.6 | 342.6 | 342.6 | 289.5 | 289.5 | 201.9 | 267.7 |
| 157.9 | 318.8 | 318.8 | 318.8 | 289.5 | 289.5 | 289.5 | 447.4 | 447.4 | 312.0 | 429.0 |
| 157.9 | 318.8 | 318.8 | 318.8 | 289.5 | 289.5 | 289.5 | 447.4 | 447.4 | 312.0 | 429.0 |
| 110.1 | 484.4 | 484.4 | 484.4 | 201.9 | 201.9 | 201.9 | 312.0 | 312.0 | 93.6 | 299.1 |
| 161.3 | 305.7 | 305.7 | 305.7 | 267.7 | 267.7 | 267.7 | 429.0 | 429.0 | 299.1 | 438.2 |

The eigenvalues are shown in Table 10.

TABLE 10
Eigenvalue (A = 1, a = −1)

| | | Contribution Ratio |
|---|---|---|
| | Eigenvalue | (Cumulative) |
| 1 | 60.201 | 0.439 (0.439) |
| 2 | 33.893 | 0.247 (0.686) |
| 3 | 22.500 | 0.164 (0.850) |
| 4 | 12.467 | 0.091 (0.941) |
| 5 | 4.203 | 0.031 (0.972) |
| 6 | 3.842 | 0.028 (1.000 |

As can be understood from comparison between Table 10 and Table 5, in case of the settings of (A=1, a=−1), the contribution ratio corresponding to the first eigenvalue is small. In this case, there is the need for the additional one eigenvalue for achieving the accumulated contribution ratio of 95% or more.

The following Table 11 shows the eigenvectors corresponding to the first and second eigenvalues.

TABLE 11
Eigenvalue (A = 1, a = −1)

| | First | Second |
|---|---|---|
| Locus | Eigenvector | Eigenvector |
| L₁ | 0.1703 | 0.1991 |
| L₂ | 0.2994 | −0.3821 |
| L₃ | 0.2994 | −0.3821 |
| L₄ | 0.2994 | −0.3821 |
| L₅ | 0.2963 | 0.3275 |
| L₆ | 0.2963 | 0.3275 |
| L₇ | 0.2963 | 0.3275 |
| L₈ | 0.3460 | 0.1254 |
| L₉ | 0.3460 | 0.1254 |
| L₁₀ | 0.2920 | −0.3891 |
| L₁₁ | 0.3373 | 0.1332 |

The distance matrix D is calculated based upon the eigenvectors. The calculation results are shown in the following Table 12.

TABLE 12
Distance (A = 1, a = −1)

| | L₂ | L₅ | L₈ | L₁₀ | L₁₁ |
|---|---|---|---|---|---|
| L₁ | 0.595 | 0.180 | 0.191 | 0.601 | 0.18 |
| L₂ | | 1.710 | 0.510 | 0.01 | 0.517 |
| L₅ | | | 0.208 | 0.717 | 0.199 |
| L₈ | | | | 0.517 | 0.012 |
| L₁₀ | | | | | 0.524 |

Subsequently, comparison is made between the distance matrix D with the settings of (A=11 a=−1) and the distance matrix with the setting of (A=0, a=1). The following Table 13 shows components of the distance matrix D with the setting of (A=0, a=1). On the other hand, the following Table 14 shows the components of the distance matrix D with the setting of (A=1, a=−1). Note that the components of the distance matrix are arrayed in order of smaller distance.

TABLE 13
Sorted distance (A = 0, a = 1)

| | Distance |
|---|---|
| d_{2,10} | 0.007 |
| d_{8,11} | 0.023 |
| d_{5,8} | 0.205 |
| d_{5,11} | 0.218 |
| d_{1,2} | 0.310 |
| d_{1,10} | 0.313 |

TABLE 14
Sorted distance (A = 1, a = −1)

| | Distance |
|---|---|
| d_{2,10} | 0.010 |
| d_{8,11} | 0.012 |
| d_{1,11} | 0.180 |
| d_{1,5} | 0.191 |
| d_{5,11} | 0.199 |
| d_{5,8} | 0.208 |

According to the present example, the distances d(2, 10) and d(8, 10) corresponding to the low-frequency haplotypes are important for selection of the representative SNPs, and as can be understood from Tables 13 and 14, the orders are the same, regardless of the settings of the alleles “A” and “a”. Accordingly, it has been confirmed that the haplotypes are omitted in order of the lower frequency. As a consequence, the representative SNPs are suitably selected.

Subsequently, description will be made regarding the haplotype frequency thereof. As can be understood from Table 2, the frequency of the fifth haplotype h₅ and the frequency of the sixth haplotype h₆ exhibit generally the same value. However, as can be understood from Table 13, in case of the settings of (A=0, a=1), there is great difference between the distance d(2, 10) and the distance d(8, 11) as compared with the relatively small difference between the frequencies of the corresponding haplotypes. On the other hand, in case of the settings of (A=1, a=−1), there is generally the same difference between the distance d(2, 10) and the distance d(8, 11) as with the difference between the frequencies of the corresponding haplotypes. Consequently, it has been confirmed that the analyzing method with the settings of (A=1, a=−1) precisely estimates the difference in the frequency of the haplotype. The pair of loci at the third smallest distance varies, depending upon the settings of the alleles “A” and “a”. However, as can be understood from comparison between the distance d(8, 11) and the next greater distance, the greater distance is approximately ten times as great as the smaller distance, regardless of the settings of (A, a). This means that the representative SNP can be selected for the third smallest distance, regardless of the settings of (A, a).

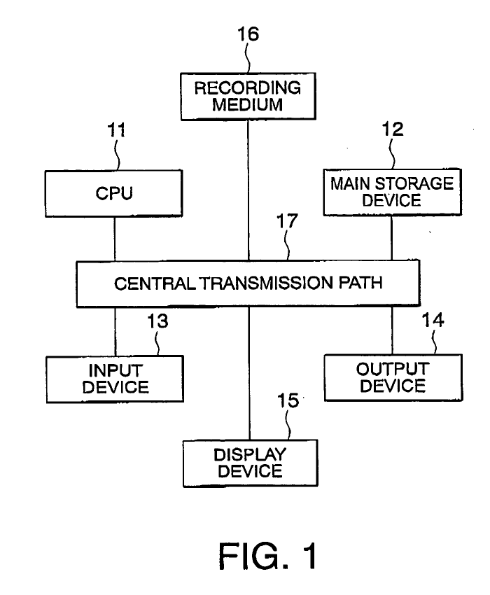

Next, description will be made of a computer system for realizing the representative SNP selection method according to the present invention with reference to FIG. 1.

As shown in FIG. 1, the computer system comprises a CPU 11, a main storage device 12, an input device 13, and output device 14, a display device 15, a recording medium 16, and a central transmission path 17 mutually connecting the above components.

The input device 13 is a device for inputting data, and comprises a keyboard, a pointing device such as a mouse, or the like. Examples of data to be input are genotypes and haplotypes. The output device 14 is a device for outputting the results obtained by the CPU 11 executing the functions thereof, and may be a printer. The display device 15 is a device for displaying results the results obtained by the CPU 11 executing the functions thereof, and may be a monitor. The recording medium 16 is a hard disk or the like.

The program for executing the representative SNP selection method according to the above-described present embodiment (hereafter also referred to as “program according to present invention”), and the input data, are saved on the storage medium 16. The CPU 11 receives signals from the input device 13, calls the program according to the present invention up to the main storage device 12, and executes the program.

First, the CPU 11 organizes the input data. If the input data is genotype data, a haplotype frequency estimation program is executed, and data according to the present invention, i.e., haplotypes, is created. If the input data is haplotypes, the input data itself is taken as the data according to the present invention.

Next, the CPU 11 calls the input data up to the main storage device 12, and executes the program according to the present invention. The results obtained thereby, i.e., the representative SNPs are displayed on the display device 15, and saved to the storage medium 16. The obtained results may also be output from the output device 14.

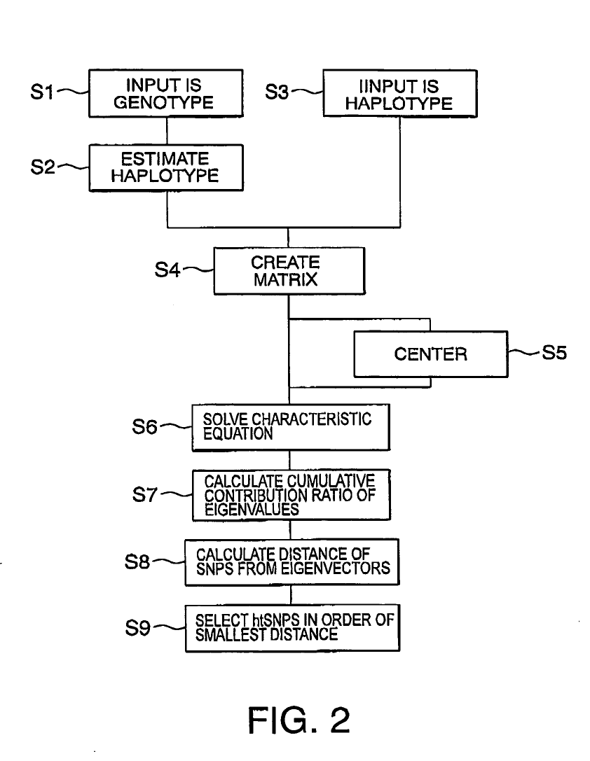

FIG. 2 is a flowchart for describing the program according to the present invention.

The input data from the input device 13 comprises genotype data and haplotype data. Let us say that input data is genotypes (step S1). In this case, the CPU 11 estimates the haplotype from the input genotype (step S2). The estimated haplotype is stored in the main storage device 12. On the other hand, let us say that the input data is haplotypes (step S3), In this event, the input haplotype is stored in the main storage device 12.

Successively, the CPU 11 creates a similarity matrix M′M from a haplotype matrix M in which each of the haplotypes serve as row vectors (Step S4), and stores this in the main storage device 12. At this time, the CPU 11 may center the haplotype matrix M (step S5).

Next, the CPU 11 solves characteristic equations (step S6), and calculates cumulative contribution ratios of eigenvalues (step S7), and further calculates the distance of SNPs from eigenvectors (step S8). The CPU 11 then selects representative SNPs (htSNPs (haplotype-tagging SNPs)) in the order of closest distance (step S9).

Therefore, steps S6 through S8 correspond to steps for obtaining association between SNPs by solving characteristic equations, and step S9 corresponds to a step for searching representative SNPs based on the association between SNPs thus obtained.

The representative SNP selection system according to the present invention is realized by the computer system shown in FIG. 1 executing the program shown in FIG. 2. Specifically, the representative SNP selection system according to the present invention is a representative SNP selection system in which a predetermined number of haplotypes can be identified from a plurality of haplotypes formed of a plurality of sets of SNPs existing close to one another on a gene in a block with strong linkage disequilibrium. The representative SNP selection system includes matrix creating means for creating a similarity matrix M′M from a haplotype matrix M in which each of a plurality of haplotypes are row vectors, association calculating means for obtaining association between SNPs by solving characteristic equations of the similarity matrix M′M, and searching means for searching for the representative SNPs from association between the obtained SNPs.

In the representative SNP selection system, the alleles of the SNPs in the haplotype matrix M may be represented by (0, 1), or by (1, −1). Herein, representing the alleles of the SNPs by (0, 1) means to represent an allele A, a as (A=0, a=1), and that representing the alleles of the SNPs by (1, −1) means to represent an allele A, a as (A=1, a=−1).

Further, the matrix creating means may use a matrix obtained by centering the haplotype matrix M upon creating the similarity matrix M′M, in order to handle variation as variance. Moreover, the association calculating means may further comprise means for calculating the cumulative contribution ratio of eigenvalues obtained by solving the characteristic equations, and means for calculating the distance between the SNPs from eigenvectors obtained by solving the characteristic equations, and the searching means may comprise means for selecting the representative SNPs in the order that the distance is closer.

While the present invention has thus far been disclosed in conjunction with several embodiments thereof, it will be readily possible for those skilled in the art to put the present invention into practice in various other manners.

1. A method for selecting representative SNPs by a computer system so as to identify a predetermined number of haplotypes from a plurality of haplotypes formed of a plurality of sets of SNPs existing close to one another on a gene in a block with strong linkage disequilibrium, comprising the steps of:
creating a similarity matrix from a haplotype matrix in which each of the haplotypes are row vectors;
obtaining association between SNPs by solving characteristic equations of the similarity matrix; and
searching for the representative SNPs from the association between the obtained SNPs.

2. A method for selecting representative SNPs according to claim 1, wherein alleles of the SNPs in the haplotype matrix are represented by (0, 1).

3. A method for selecting representative SNPs according to claim 1, wherein alleles of the SNPs in the haplotype matrix are represented by (1, −1).

4. A method for selecting representative SNPs according to claim 1, wherein a matrix obtained by centering the haplotype matrix is used upon creating said similarity matrix.

5. A method for selecting representative SNPs according to claim 1, wherein a step for obtaining association between SNPs further comprising the steps of;
calculating a cumulative contribution ratio of eigenvalues obtained by solving the characteristic equations, and
calculating a distance between the SNPs from eigenvectors obtained by solving the characteristic equations; and
a step for searching for representative SNPs comprising a step for selecting the representative SNPs in the order that the distance is closer.

6. A representative SNP selection system so as to identify a predetermined number of haplotypes from a plurality of haplotypes formed of a plurality of sets of SNPs existing close to one another on a gene in a block with strong linkage disequilibrium, comprising:
matrix creating means for creating a similarity matrix from a haplotype matrix in which each of the haplotypes are row vectors;
association calculating means for obtaining association between SNPs by solving characteristic equations of the similarity matrix; and
searching means for searching for the representative SNPs from the association between the obtained SNPs.

7. A representative SNP selection system according to claim 6, wherein alleles of said SNPs in the haplotype matrix are represented by (0, 1).

8. A representative SNP selection system according to claim 6, wherein alleles of the SNPs in the haplotype matrix are represented by (1, −1).

9. A representative SNP selection system according to claim 6, wherein a matrix obtained by centering the haplotype matrix is used upon creating the similarity matrix.

10. A representative SNP selection system according to claim 6, the association calculating means further comprising;
means for calculating a cumulative contribution ratio of eigenvalues obtained by solving the characteristic equations, and
means for calculating a distance between the SNPs from eigenvectors obtained by solving the characteristic equations; and
the searching means comprising means for selecting the representative SNPs in the order that the distance is closer.

11. A representative SNP selection program in which a computer identifies a predetermined number of haplotypes from a plurality of haplotypes formed of a plurality of sets of SNPs existing close to one another on a gene in a block with strong linkage disequilibrium, said program causing said computer to execute the following functions:
a matrix creating function for creating a similarity matrix from a haplotype matrix in which each of the haplotypes are row vectors;
an association calculating function for obtaining association between SNPs by solving characteristic equations of the similarity matrix; and
a searching function for searching for said representative SNPs from the association between the obtained SNPs.

12. A program according to claim 11, wherein alleles of said SNPs in the haplotype matrix are represented by (0, 1).

13. A program according to claim 11, wherein alleles of said SNPs in the haplotype matrix are represented by (1, −1).

14. A program according to claim 11, wherein a matrix obtained by centering the haplotype matrix is used upon creating the similarity matrix.

15. A program according to claim 11, the association calculating function further comprising;
a function for calculating a cumulative contribution ratio of eigenvalues obtained by solving said characteristic equations, and
a function for calculating a distance between the SNPs from eigenvectors obtained by solving the characteristic equations; and
the searching function comprising a function for selecting the representative SNPs in the order that the distance is closer.
